(19) United States
(12) Patent Application Publication
(10) Pub. No.: US 2003/0109841 A1
(43) Pub. Date: Jun. 12, 2003

(54) Disposable diaper with auxiliary pockets

(76) Inventor: Lawrence A. Edwards, Bloomington, CA (US)

Correspondence Address: GARY R. ILMANEN, THE LAW OFFICES OF GARY R. ILMANEN, 3992 BROCKTON AVENUE, RIVERSIDE, CA, 92501-3203, US

(21) Appl. No.: 10/013,248

(22) Filed: Dec. 10, 2001

(51) Int. Cl. A61F 13/15

(52) U.S. Cl. 604/385.06

(57) ABSTRACT

An improved disposable diaper is provided: an absorbent article adapted to be worn about the lower torso of a wearer. The diaper portion is of customary design. The article features a novel re-useable pocket, or pockets, for the storage of small items, such as identity cards, money, prepackaged moist towellettes/ointment/medication, and/or small personal possessions. A panel is generally attached by its edges to the exterior of the article, forming a closed space between the article and the panel. An opening is defined to provide access to the enclosed space, and a closure provides security for the contents. By folding the panel prior to attachment, a second enclosed space with integral closure flap is formed within the panel.

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO A MICROFICHE APPENDIX

Not Applicable.

### BACKGROUND OF THE INVENTION

The present invention relates to diapers, and more specifically, to novel disposable diapers equipped with storage pockets.

Diapers have been used for many years to protect garments from inadvertent release of bodily waste products. Disposable absorbent articles having many different basic designs are known in the art. Since their introduction, disposable diapers have become much more popular than the traditional type of cloth diaper. Because of the disposable's rapid attachment and release, caregivers have virtually abandoned the use of the cloth diaper and safety pin. Adding to its attractiveness is freedom from the necessity of carrying soiled diapers home to be laundered. U.S. Reissue Pat. No. Re. 26,152, entitled “Disposable Diaper,” which issued on Jan. 31, 1967, to Duncan et al., describes a basic disposable diaper structure that has achieved wide acceptance and considerable commercial success. The disposable diaper has continuously evolved and improved since its introduction.

Paralleling the improvements in the disposable diaper, the diaper bag has also evolved. This accessory is used when away from home, providing a convenient place to store cans of powder, moist towelettes, ointment, oil and so forth.

The present invention is intended to provide a convenient reusable storage pocket as a part of the diaper. This pocket may be used to store small items, to insert an identity card, or to free the caregiver from having to carry bulky containers of diaper bag necessities. This is accomplished by providing a space to store individual-use packages of such products where it is needed most: right there with the end user.

Generally, prior art garments containing pockets are targeted specifically for adults. For example, in U.S. Pat. No. 5,093,935, Countee teaches an undergarment which include a centrally-located pocket in the front for the storage of small items, such as prophylactics. Marsh, in U.S. Pat. No. 4,852,188, discloses a panty-type garment with a security pocket for valuables. U.S. Pat. No. D387,538 (Taylor) and U.S. Pat. No. D396,542 (Nicholson) both show ornamental designs for a type of pocketed underwear, allowing the wearer to carry items such as prophylactics. U.S. Pat. No. D358,472 (Firouzpour) shows the ornamental design for a combined brief and condom pockets. U.S. Pat. No. 4,533,355 (Fair) teaches an oversized, loose-fitting undergarment for individuals wearing an ostomy, containing a pocket on the exterior with a cover flap to shield the appliance from view.

It appears that the cited prior art teaches undergarment pocket techniques with a view toward hiding items of value or items that may be a cause of embarrassment. Until now, no one has proposed combining a re-usable pocket with a diaper, because babies have no use for a means to hide valuables, they are not easily embarrassed, and most do not have the dexterity to make use of such an item.

Over the years, pockets have been attached to innumerable items. Most recently, a close reference to the present invention is found in U.S. Pat. No. 6,000,056, which teaches a disposable bib adapted to carry articles for use in feeding. Here, Brady has developed a protective bib that contains a pocket in which eating utensils may be stored until ready for use. Similarly, in U.S. Pat. No. 4,221,221 Ehrlich shows a disposable diaper incorporating powder, towel, and oil packets. U.S. Pat. No. 4,738,678, U.S. Pat. No. 4,931,052, U.S. Pat. No. 5,304,158, and U.S. Pat. No. 6,004,307, are variations of Ehrlich's theme.

However, none of the combinations teach a re-usable, closable, resealable pocket, as is used in the present invention. All of the prior art containers cited are intended for single-use, and are essentially destroyed upon opening.

### BRIEF SUMMARY OF THE INVENTION

In accordance with one aspect of the present invention, there is provided an absorbent article that is adapted to frictionally engage the torso of the individual about the waist. The article includes a liquid impervious backsheet having an outer-facing surface and an inner body-facing surface. An absorbent core is provided adjacent the body-facing surface of the backsheet. The absorbent core has a shape to enable the core to be placed adjacent the crotch area of the body of a wearer and has the capacity to absorb liquids. A flexible, liquid-pervious topsheet overlies the absorbent core. The article includes storage pockets integral to or attached upon the backsheet, accessible from the exterior.

The preferred embodiment has two individual pockets, one behind the other. The first pocket utilizes a closure that interferes with an infant's access to the contents. The second pocket is for further convenience. The pockets may be used to store prepackaged moist towelettes, powder ointment, medication or any small accessory. The caregiver's business card or other identification could also be placed in the pocket to aid in identification and recovery of a lost child.

The pockets, as well as the packages they contain, add no noticeable weight or discomfort to the user, leaving them out of sight and mind. While the infant version proposes that the pockets be located on the rear surface, an adult version may have the pocket in the front or side for convenience.

As noted, security for the contents is provided by a closure. The closure can be effected by way of a fold-over design, a zipper, a Ziploc™ or a Velcro™ closure, among others. In one embodiment, an adhesive patch pocket may be applied over the items to be stored.

In summary, the pockets on the diaper gives the caregiver the convenience of keeping handy or essential materials close by, and reduces or eliminates the need to carry bulky containers of supplies. This novel concept of diapers with pockets can be incorporated into all forms of existing disposable diaper technology. Caregivers can load the pockets with the desired materials, and/or the diaper pockets can be preloaded with prepackaged materials at the manufacturing stage, prior to sale to the consumer.

### BRIEF DESCRIPTION OF THE SEVERAL VIEWS OF THE DRAWING

FIG. 1 is a plan view of a strap-on type of disposable diaper with pockets arrangement according to the present invention, showing the surface intended to be worn facing away from the body. FIG. 2 is a side view of the same item. FIG. 3. is sectional view I-I of FIG. 1.

### DETAILED DESCRIPTION OF THE INVENTION

Details of a disposable diaper with pockets according to the invention will be more fully understood from the description given hereunder with reference to the accompanying drawings.

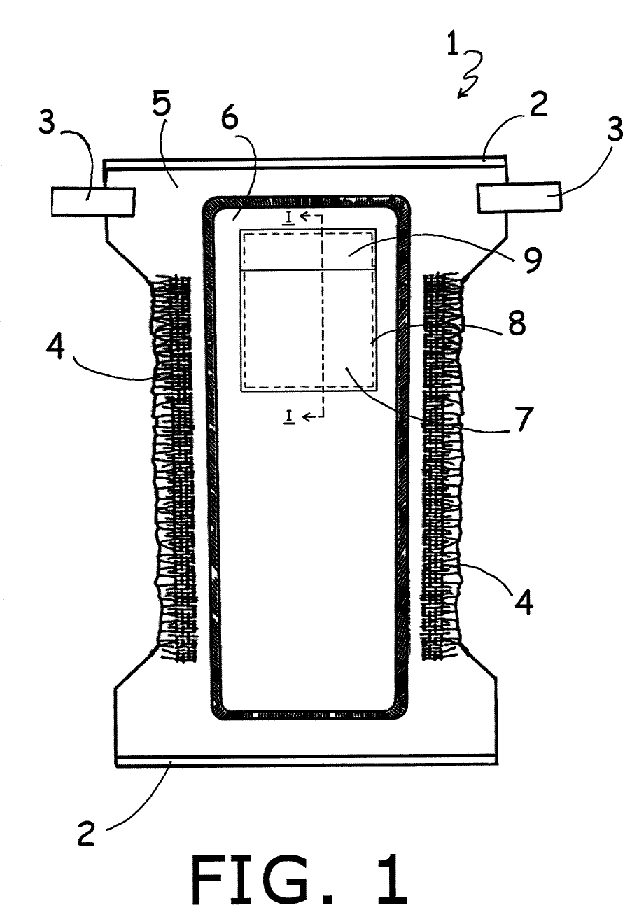

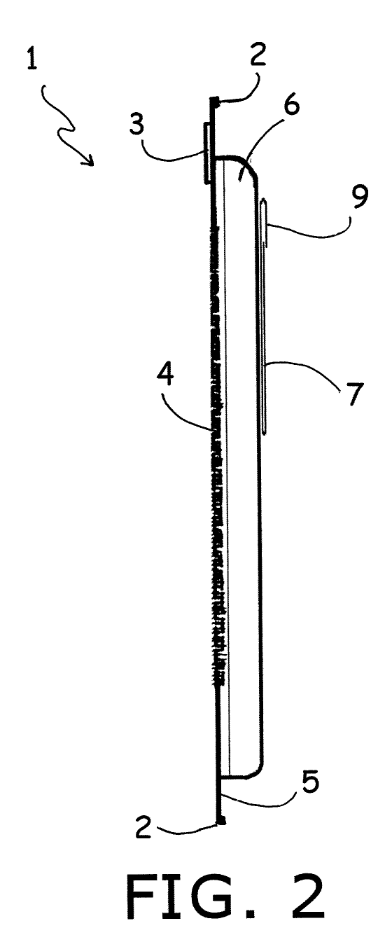

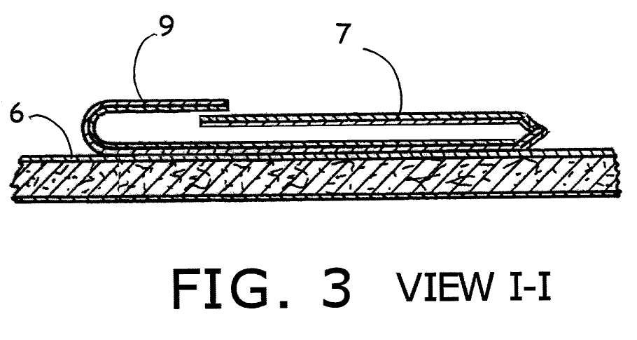

FIG. 1 is a plan view of the preferred embodiment of the present invention, in the form of a strap-on style of disposable diaper. The depiction of a strap-on style diaper is for illustration only; the present invention may be applied to the pull-up style, or any other style of disposable diaper. The diaper 1 is of customary design, including a waist band 2, which is preferably of an elastic material, and which defines a waist opening when the diaper is deployed such that the two waist bands 2 are brought together and secured with adhesive tabs 3. The diaper 1 further includes elastic fabric tapes 4 that define leg openings when the diaper is deployed.

In the preferred embodiment of the invention, the liquid impervious backsheet 5 covers the absorbent core within the diaper 1, thereby forming a raised area 6 at the rear of the diaper 1. The panel 7 is a piece of material which is folded twice as shown in FIG. 2, then generally attached to liquid impervious backsheet 5 by its left, right, bottom and optionally top edges to the outward-facing surface of diaper 1, such that it extends proximally from below waist band 2 to an area between elastic fabric tapes 4. Panel 7 may be attached by simple stitched seams 8, adhesive, thermal bonding, or other bonding processes. The left and right attachment points for panel 7 may be coextensive with the edges of elastic fabric tapes 4.

Defined in panel 7 proximally below waist band 2 is the uppermost folded portion of panel 7, comprising closure flap 9, which defines an opening to the enclosed space formed within the folded panel 7. This opening at the bottom of closure flap 9 provides access to the space formed within panel 7, creating a storage pocket suitable for small items. Thus, the items deposited within the pocket remain accessible through the opening at the bottom of closure flap 9, yet are not prone to fall out. Closure flap 9 is designed to be self-closing, but may be secured additionally by any means of closure, such as a zipper, a Ziploc™ fastener, a Velcro™ hook-and-loop fastener, button, etc.

In one embodiment, panel 7 is not secured to liquid impervious backsheet 5 on its top edge; as panel 7 is attached only by its left, right, and bottom edges, a secondary enclosed space is formed between the liquid impervious backsheet 5 and Panel 7. The assembly therefore comprises two separated pockets, with unfettered access to the secondary pocket through the resulting top opening formed between panel 7 and liquid impervious backsheet 5. This secondary storage pocket obviously has no retaining flap, but may be secured by replacing the uppermost portion of seam 8 with any means of closure, such as a zipper, a Ziploc™ fastener, a Velcro™ hook-and-loop fastener, button, etc.

In another embodiment, the enclosed space is formed by attaching the panel 7 to the body-facing surface of the liquid impervious backsheet 5 such that the compartment is integrally formed within the diaper 1 and accessible through a slit suitably located on the liquid impervious backsheet 5 and piercing same. The resulting enclosed space may be sealed with a slit closuring means of any type in order to inhibit access to or loss of the items stored therein.

In another embodiment, folded panel 7 is stitched with stitches 8, then adhesive material is applied to the top, left, right, and bottom edges of the panel 7. The secondary enclosed space is formed by attaching panel 7 to the outward-facing surface of the liquid impervious backsheet 5 such that the contents are trapped securely, interstitially, at the time of attachment. No opening is provided; access is obtained by removing the entire panel 7.

In another embodiment, two or more panels 7 are attached in a stacked manner to liquid impervious backsheet 5 to define multiple enclosed spaces.

Each of the foregoing embodiments may include multiple placements of panel 7 on various surfaces of the backsheet 5, including the front, rear, and sides. The novelty of the present invention lies in the feature of a re-usable pocket or pockets attached to, and made a part of, the disposable diaper.

It will be readily understood by those persons skilled in the art that the present invention is susceptible of a broad utility and application. Many embodiments and adaptations of the present invention other than those herein described, as well as many variations, modifications and equivalent arrangements will be apparent from or reasonably suggested by the present invention and the forgoing description thereof, without departing from the substance, scope, spirit or essential attributes of the present invention.

Accordingly, while the present invention has been described herein in detail in relation to its preferred embodiment, it is to be understood that this disclosure is only illustrative and exemplary of the present invention and is made merely for purposes of providing a full and enabling disclosure of the invention. The foregoing disclosure is not intended to limit, nor is it to be construed to as limiting, the present invention or otherwise to exclude any such other embodiments, adaptations, variations, modifications and equivalent arrangements, the present invention being limited only by the claims appended hereto and the equivalents thereof.

What I claim as my invention is:

1. A disposable diaper comprising:
a liquid-pervious flexible topsheet adapted to face the body of the wearer,
a flexible absorbent core positioned proximally to said topsheet,
a liquid-impervious flexible backsheet positioned proximally to the opposite surface of said core, said backsheet adapted to face outwardly relative to the body a wearer,
a rectangular panel of material having means for affixing in overlying relationship to said backsheet, said panel defining a first pocket formed between said panel and said backsheet.

2. The disposable diaper of claim 1, further comprising a closure means extending across the opening of said first pocket.

3. The disposable diaper of claim 1, said panel containing a fold line extending from the left edge of said panel to the right edge of said panel, said fold line located approximately 30 to 40 percent of the height of said panel, said fold line defining a smaller and a larger panel surface, joined at the bottom, and containing a second fold line extending from the left edge of said larger panel surface to the right edge of said larger panel surface, said second fold line located approximately 80 to 90 percent of the height of said panel, said second fold line defining a closure flap joined to the larger panel surface at the top and partially overlying the short edge surface of the said smaller panel section, the said larger panel surface affixed to said backsheet, thereby defining a second pocket formed in overlying relationship to said first pocket.

4. The disposable diaper of claim 1, said panel attached to the opposite surface of said backsheet and disposed between said backsheet and said core, forming a first pocket interstitially therein,
a slit piercing said backsheet providing access to said first pocket,
a closure means securing said slit.

5. The disposable diaper of claim 1, further comprising:
an item or items releasably received within said first or second pocket, said item or items comprising a business card; an identification card; a driver's license; personal possessions of an aggregate size smaller than said first or second pocket; a prepackaged moist towelette, powder, and/or ointment; money; credit card; food; candy; and medicine.

6. The invention of claim 1 further comprising a plurality of panels of material having means for affixing in overlying relationship to said backsheet, said panels defining multiple pockets.
